(19) United States
(12) Patent Application Publication
(10) Pub. No.: US 2020/0383655 A1
(43) Pub. Date: Dec. 10, 2020

(54) ACTION INSTRUCTION DEVICE AND X-RAY IMAGING APPARATUS EQUIPPED WITH THE SAME

(71) Applicant: Shimadzu Corporation, Kyoto (JP)

(72) Inventors: Hideki FUJII, Kyoto (JP); Akiharu YAMAGATA, Kyoto (JP)

(21) Appl. No.: 16/861,892

(22) Filed: Apr. 29, 2020

(30) Foreign Application Priority Data
Jun. 5, 2019 (JP) 2019-105400

(51) Int. Cl. A61B 6/00

(52) U.S. Cl. CPC A61B 6/541; A61B 6/463; A61B 6/50; A61B 6/42; A61B 6/52; A61B 6/40

(57) ABSTRACT

The present invention relates to an action instruction device and an X-ray imaging apparatus equipped with the same. A table stores a plurality of pieces of breath action instruction data corresponding to a plurality of combinations of an imaging region and an examination type, respectively. A speaker and a display instruct a breath action according to breath action instruction data. The breath action instruction data corresponding to the combination of the imaging region and the examination type is acquired by a control unit from the table. With this, it may be possible to prevent a setting error of instruction data for a breath action corresponding to a predetermined combination of an imaging region and an examination type.

This application claims priority under 35 U.S.C. § 119 to Japanese Patent Application No. 2019-105400 filed on Jun. 5, 2019, the entire disclosure of which is incorporated herein by reference in its entirety.

### BACKGROUND OF THE INVENTION

### Technical Field

The present invention relates to an action instruction device for instructing a breath action to a subject, and also relates to an X-ray imaging apparatus for performing X-ray imaging on a subject, the X-ray imaging apparatus being equipped with the action instruction device.

### Background of the Invention

An X-ray imaging apparatus is provided with an X-ray irradiation unit for emitting X-rays, an X-ray detector (X-ray receiving unit) for detecting X-rays, an imaging table mounting the X-ray irradiation unit and the X-ray detector, and a control unit for controlling the entire apparatus. The X-ray irradiation unit is provided with an X-ray tube, an X-ray tube holding unit for holding the X-ray tube, a collimator for controlling the irradiation field of the X-rays emitted from the X-ray tube, and a high voltage generating unit for generating a high voltage for a tube voltage or a tube current for the X-ray tube. The imaging table is composed of a table for placing a subject horizontally thereon, a stand for holding a subject with a standing posture, or a combination of a table and a stand. In some cases, an X-ray imaging apparatus is provided with a moving mechanism for moving an X-ray irradiation unit and an X-ray detector.

Generally, a radiology department information system (RIS: Radiology Information System) issues an examination order to an image processing unit when examining a subject to be imaged. Note that the radiology department information system is hereinafter referred to as “RIS”. Also note that, an image processing unit is hereinafter referred to as a “DR system (digital radiography system)”. An examination order includes, for example, information on a subject (name, sex, age, etc.) and X-ray imaging menus. In some cases, a plurality of X-ray imaging menus (chest, lungs, abdomen, etc.,) is scheduled for one subject.

When a technician (operator) of an X-ray imaging apparatus selects an examination order from a console of a DR system, the X-ray imaging apparatus (or X-ray imaging apparatus's main body) is notified of the information about the subject and the imaging condition corresponding to the expanded X-ray imaging menu. After completing all X-ray imaging scheduled for the examination, the technician notifies the RIS of the completion of the examination and transfers the X-ray image (captured image) acquired by the imaging to a server.

Imaging of each of a chest, a lung, an abdomen, etc., is performed with a subject in a predetermined breath condition to acquire an image appropriate for diagnosis. For example, chest imaging is performed in a state in which a breath is held (state maintenance). Imaging of a lung region is performed with breath held in an exhaled state. Imaging of an abdomen region is performed with breath held in an inhaled state. In this manner, a different breath action is instructed to a subject depending on X-ray imaging.

A breath action (e.g., an exhalation action, an inhalation action, or a state maintenance action) to a subject is sometimes made by a verbal instruction of a technician. A breath action may be performed by an instruction through a speaker or a display. For example, a function of automatically issuing a voice instruction to a subject by a speaker in synchronism with an imaging operation of a technician is called an “auto voice function”. For example, a breath action instruction is issued by an imaging hand switch (exposure switch) when the imaging preparation is initiated, and for example, an instruction to release the state maintenance is issued when the imaging is completed.

To an X-ray imaging apparatus, a medical-use voice generation device having an auto voice function is externally attached, or an X-ray imaging apparatus has a built-in voice generation device.

As this type of medical-use voice generation device, for example, there is a device described in Patent Document 1. This device performs control to match the timing of holding the subject's breath with the timing of outputting audio guidance as the timing of capturing an image of the subject.

### PRIOR ART DOCUMENT

### Patent Document

Patent Document 1: Japanese Unexamined Patent Application Publication No. 2008-067935

### SUMMARY OF THE INVENTION

### Problems to be Solved by the Invention

However, a conventional example having such a configuration has the following problems.

A conventional device performs imaging aimed at an exhaled or inhaled states depending on an imaging region. At this time, the order of breath actions is manually set on an operation panel of a medical voice generation device by a technician. If the manual setting is incorrect, a subject performs a breath corresponding to the incorrect setting. As a result, the imaging operation needs to be performed again, and therefore there is a problem that the load on the technician and the subject increases. Further, there is also a problem that the examination time is extended by the redo of the imaging operation, which lowers the throughput of the examination.

The present invention has been made in view of the above circumstances, and an object thereof is to provide an action instruction device capable of preventing a setting error of a breath action and an X-ray imaging apparatus equipped with the action instruction device.

### Means for Solving the Problem

In order to achieve the above object, the present invention has the following configuration.

That is, an action instruction device includes:

an instruction data storage unit configured to store a plurality of pieces of breath action instruction data corresponding to a plurality of combinations of an imaging region and an examination type, respectively, the plurality of pieces of breath action instruction data each including at least one of an exhalation action, an inhalation action, and a state maintenance action;

a control unit configured to acquire the breath action instruction data corresponding to a received combination of an imaging region and an examination type upon receipt of the combination of the imaging region and the examination type from outside; and
an instruction execution unit configured to instruct a breath action in accordance with the breath action instruction data acquired by the control unit.

Further, an X-ray imaging apparatus for performing X-ray imaging according to the present invention includes:

an input unit configured to input a combination of an imaging region and an examination type;

an X-ray irradiation unit configured to emit X-rays;

an X-ray detector configured to detect X-rays;

an image processing unit configured to process an image composed of a pixel value of X-rays detected by the X-ray detector to generate an X-ray image; and

an action instruction device configured to instruct a breath action to a subject,

wherein the action instruction device includes:

an instruction data storage unit configured to store a plurality of pieces of breath action instruction data corresponding to a plurality combinations of an imaging region and an examination type, respectively, the plurality of pieces of breath action instruction data each including at least one of an exhalation action, an inhalation action, and a state maintenance action;

a control unit configured to acquire the breath action instruction data corresponding to a received combination upon receipt of the combination of the imaging region and the examination type from the input unit; and

an instruction execution unit configured to instruct a breath action according to the breath action instruction data acquired by the control unit.

### Effects of the Invention

According to the action instruction device of the present invention and the X-ray imaging apparatus equipped with the same, the instruction data storage unit stores a plurality of pieces of breath action instruction data corresponding to a plurality of combinations of an imaging region and an examination type. The instruction execution unit instructs a breath action according to the breath action instruction data. The breath action instruction data corresponding to the combination of the imaging region and the examination type is acquired by the control unit from the instruction data storage unit. That is, the control unit acquires the optimal breath action instruction data corresponding to the combination of the imaging region and the examination type. With this, it is possible to prevent a setting error of a breath action corresponding to a predetermined combination of an imaging region and an examination type.

### BRIEF DESCRIPTION OF THE DRAWINGS

FIG. 1 is a schematic perspective view of an X-ray imaging apparatus composed of an upright stand according to respective embodiments.

FIG. 2 is a block diagram of an X-ray imaging apparatus and a medical voice generation apparatus according to Embodiment 1.

FIG. 3A is a diagram showing an example of an association between a combination of an imaging region and an examination type and breath action instruction data, and FIG. 3B is a diagram for explaining how breath action instruction data is acquired in accordance with a combination of an imaging region and an examination type of each examination order.

FIG. 4 is a diagram showing an example of a screen displayed on an operation display for checking and changing a breath action.

FIG. 5 is a block diagram of an X-ray imaging apparatus and a medical voice generation device according to Embodiment 2.

### DETAILED DESCRIPTION OF THE PREFERRED EMBODIMENTS

### Embodiment 1

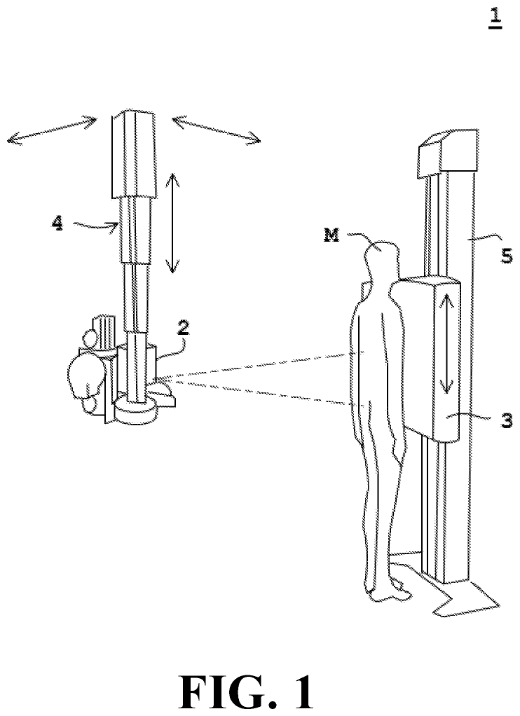

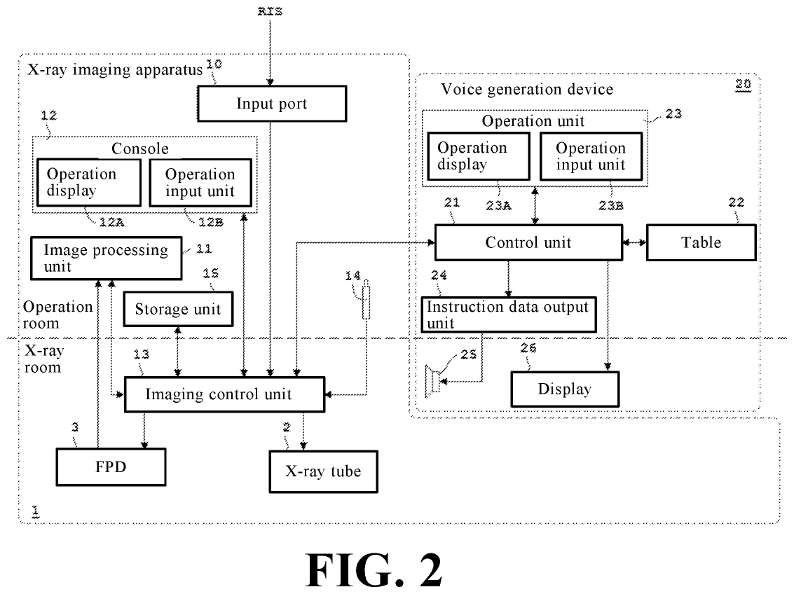

Hereinafter, with reference to the attached drawings, Embodiment 1 of the present invention will be described. FIG. 1 is a schematic perspective view of an X-ray imaging apparatus composed of an upright stand according to an embodiment. FIG. 2 is a block diagram of an X-ray imaging apparatus and a medical-use voice generation device (hereinafter referred to as “voice generation device”) according to Embodiment 1. In this Embodiment 1, including Embodiment 2 to be described later, a flat-panel X-ray detector (FPD: Flat Panel Detector) will be exemplified as an X-ray detector. FIG. 1 shows a configuration common to each embodiment.

### <Configuration of X-ray Imaging Apparatus 1>

Reference is made to FIG. 1. An X-ray imaging apparatus 1 is provided with an X-ray tube 2, a flat-panel X-ray detector 3 (hereinafter referred to as “FPD”), a column 4, and an upright stand 5.

The X-ray tube 2 emits X-rays. The FPD 3 detects X-rays emitted from the X-ray tube 2 and passed through the subject M. A collimator (not shown) for controlling the irradiation field of the X-rays is provided on the radiation side of the X-ray tube 2. The X-ray tube 2 is supported by the column 4 and is configured to be adjustable in orientation. The column 4 is movable (capable of traveling) along rails (not shown) provided on the ceiling. The column 4 is configured to be extendable and retractable. Since the X-ray tube 2 is supported by the column 4, the X-ray tube 2 is horizontally/vertically movable.

The upright stand 5 supports a subject M with a standing posture. The upright stand 5 is installed on the floor. The FPD 3 is mounted on the upright stand 5 and is configured to be movable up and down. When capturing an image of an imaging region of a chest, a lung, an abdomen, or the like, positioning (alignment) is performed so that the FPD 3 is positioned to the imaging region (e.g., chest) by moving the FPD 3 up and down. The direction of the X-ray tube 2 is adjusted so that the X-rays from the X-ray tube 2 are irradiated onto the FPD 3 positioned at the imaging region, and the column 4 is moved to expand and contract so that the X-ray tube 2 is positioned at a position facing the imaging region. Note that the direction of the X-ray tube 2 can also be adjusted so that the X-rays from the X-ray tube 2 are irradiated onto a table (imaging table) (not shown) on which a subject M is mounted horizontally.

Reference is made to FIG. 2. The X-ray imaging apparatus 1 is further provided with an input port 10, an image processing unit 11, a console 12, an imaging control unit 13, an exposure switch 14, and a storage unit 15.

The input port 10 is configured to accept an examination order from the RIS. The input port 10 is composed of, for example, terminals and electric circuits when an examination order is received by wire. The input port 10 is composed of, for example, antennas and electric circuits when an examination order is received wirelessly. The examination order includes subject information (name, sex, age, etc.) of a subject and one or a plurality of X-ray imaging menus.

The X-ray imaging menu contains information on the imaging region (chest, lungs, abdomen, etc.) and examination type data. The examination type is information for distinguishing the type of examination (X-ray imaging). For example, the examination type may be composed of letters, symbols, numbers, or combinations thereof. The examination type may be information on, e.g., the imaging direction and the body thickness. The examination type may be information on an imaging region, an imaging direction, and a body thickness, in addition to an imaging region.

The image processing unit 11 processes the image composed of a pixel value of X-rays detected by the FPD 3 to generate an X-ray image. The console 12 is configured to allow a technician to set and change, for example, imaging conditions. The console 12 is provided with an operation display (monitor) 12A and an operation input unit 12B. The operation display 12A is composed of, for example, a liquid crystal display or an organic EL (organic electro-luminescence) display. The operation input unit 12B is provided with at least one of a plurality of switches, a touch panel, a keyboard, and a mouse. The touch panel is composed of, for example, a resistive film type touch panel, and is arranged on the front surface of the operation display 12A.

The imaging control unit 13 performs overall control with respect to imaging. The image processing unit 11, the imaging control unit 13, and a control unit 21 which will be described later are each composed of a central processing unit (CPU; Central Processing Unit) and the like. Note that the image processing unit 11 may be configured by circuits, such as a GPU (Graphics Processing Unit).

The exposure switch 14 performs irradiation preparation/irradiation initiation when being pressed. The exposure switch 14 is composed of a two-stage switch. When a technician presses the exposure switch 14 halfway (first step pressing), a tube voltage is applied to the X-ray tube 2 to set the radiation preparation. When the technician fully presses the exposure switch 14 (second step pressing), X-ray irradiation from the X-ray tube 2 is initiated.

Note that the X-ray irradiation from the X-ray tube 2 is not initiated at the same time when the exposure switch 14 is fully pressed. In either imaging region, since the imaging is performed in a state in which a breath is held (while maintaining the state), the X-ray irradiation from the X-ray tube 2 is initiated after a preset time has elapsed from the time when the exposure switch 14 is fully pressed. On the other hand, at the timing when the exposure switch 14 is fully pressed, the control unit 21 outputs instruction data corresponding to the X-ray imaging menu in order to send instruction data to the speaker 25 and the display 26. Note that the instruction data may include the timing of starting a breath action and information, such as, e.g., a length of voice guidance for the breath action.

The storage unit 15 stores a plurality of examination orders received from the RIS. The storage unit 15 stores the imaging condition corresponding to the X-ray imaging menu of the examination order. The storage unit 15 also stores programs for operating the X-ray imaging apparatus 1. The storage unit 15 and a table 22 which is described later are composed of a storage medium typified by a RAM (Random-Access Memory) or the like.

### <Configuration of Voice Generation Device 20>

The X-ray imaging apparatus 1 is further provided with a voice generation device 20 as an external device. The voice generation device 20 is provided with a control unit 21, a table 22, an operation unit 23, an instruction data output unit 24, a speaker 25, and a display 26.

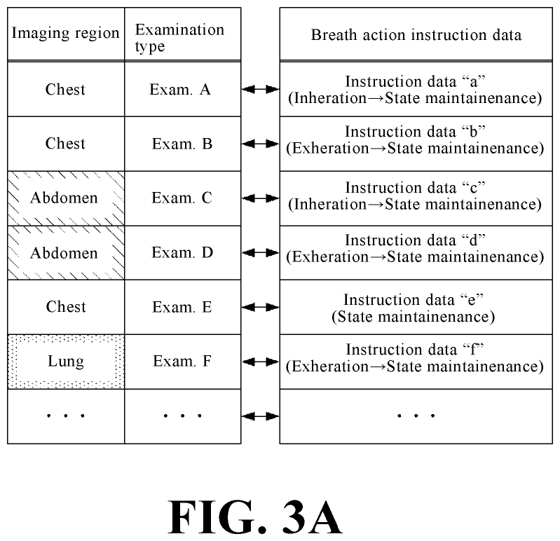

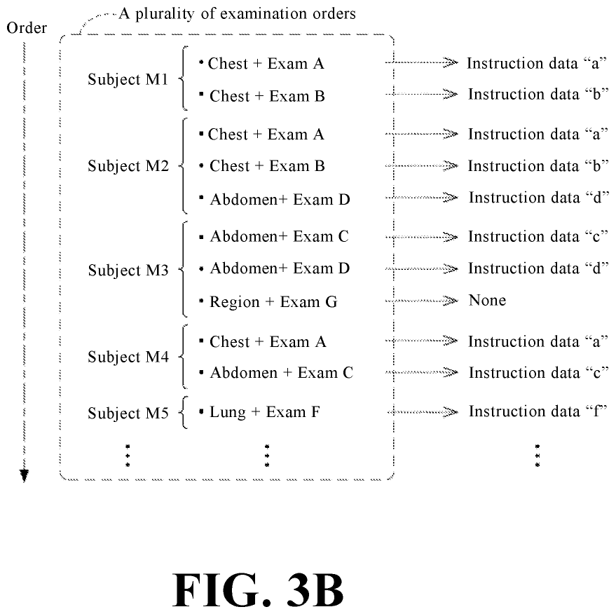

The control unit 21 performs overall control concerning sound. The table 22 stores a plurality of pieces of breath action instruction data corresponding to a plurality of combinations of an imaging region and an examination type, respectively, as shown in FIG. 3A. That is, the table 22 stores a predetermined combination of an imaging region and an examination type and predetermined breath action instruction data in association with each other. The breath action instruction data is hereinafter referred to as “instruction data” as appropriate. The instruction data is preset data.

FIG. 3A is a diagram illustrating an exemplary association between a combination of an imaging region and an examination type and instruction data. In FIG. 3A, for example, the instruction data “a” corresponds to a combination of “Chest and Examination A”. The instruction data “c” corresponds to a combination of “Abdomen and Examination C”. The instruction data “e” corresponds to the combination of “Chest and Examination E”. The instruction data “f” also corresponds to a combination of “Chest and Examination F”. The instruction data “a” to “f” each include at least one of an exhalation action, an inhalation action, and a state maintenance action. The instruction data will be described in the descriptions of the speaker 25 and the display 26.

Upon receiving a combination of an imaging region and an examination type (e.g., Chest and Examination A) from the input port 10 or the console 12, the control unit 21 acquires instruction data “a” corresponding to the received combination (e.g., Chest and Examination A) from the table 22. The contents of a predetermined examination (for example, Examination A) may be the same as the content of the other examination (for example, Examination B).

FIG. 3B is a diagram for explaining a state in which instruction data is acquired in accordance with a combination of an imaging region and an examination type of each examination order. In FIG. 3B, it is assumed that X-ray imaging is performed in the order of Subject M1, Subject M2, Subject M3, Subject M4, and Subject M5. For example, the examination order for Subject M1 has two X-ray imaging menus. The first X-ray imaging menu includes information about “Chest and Examination A:. The second X-ray imaging menu includes “Chest and Examination B”. The examination order for Subject M5 includes one X-ray imaging menu (including “Lung and Examination F”).

The control unit 21 acquires the instruction data corresponding to each combination (e.g., “Chest and Examination A”) of the imaging region and the examination type of a plurality of X-ray imaging menus from the table 22 to perform the X-ray imaging in order based on the plurality of X-ray imaging menus. In the case of the X-ray imaging of “Chest and Examination A” for Subject M1, the control unit 21 acquires the instruction data “a” from the table 22. In the case of the X-ray imaging of “Chest and Examination B” for Subject M1, the control unit 21 acquires the instruction data “b” from the table 22. Further, in the case of the X-ray imaging of “Lung and Examination F” for Subject M5, the control unit 21 acquires the instruction data “f” from the table 22.

Note that in FIG. 3B, for example, in cases where no guidance of a breath action is required as in the case of the X-ray imaging of a predetermined imaging region and Examination G for Subject M3, the control unit 21 may be configured to not acquire instruction data.

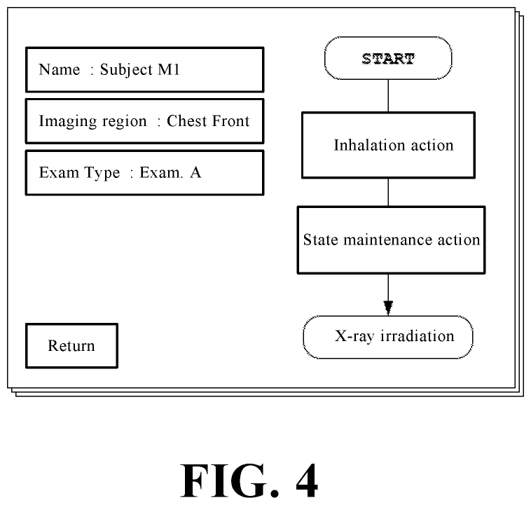

Returning to FIG. 2, the operation unit 23 is composed of, for example, an operating panel for selecting a breath action. The operation unit 23 is provided with an operation display 23A and an operation input unit 23B. The operation display 23A is composed of, for example, a liquid crystal display or an organic EL display. As shown in FIG. 4, the operation display 23A shown in FIG. 2 displays a part or all of the contents of the X-ray imaging menu (the examination type, the imaging region, and the like). The operation display 23A also displays the instruction data. In FIG. 4, for example, the name of Subject M1, the name of the imaging region, and the name of Examination A are displayed on the operation display 23A. Further, the instruction data is displayed on the operation display 23A. The instruction data to be displayed includes, for example, an inhalation action and a state maintenance action are performed in this order.

Returning to FIG. 2, the operation input unit 23B is configured to be able to change the instruction data displayed on the operation display 23A. The operation input unit 23B is composed of, for example, a plurality of switches or a touch panel. The plurality of switches is arranged around the operation display 23A. The touch panel is composed of, for example, a resistive film type touch panel, and is arranged on the front surface of the operation display 23A.

The instruction data output unit 24 is composed of an electric circuit including any one of an amplifier and a digital-to-analog converter (D/A converter). The instruction data output unit 24 amplifies instruction data of a breath action and/or performs digital-to-analog conversion, and then outputs the instruction data of the breath action. That is, the instruction data outputting unit 24 sends the amplified and digital-to-analog converted breath action instruction data to the speaker 25, for example.

The speaker 25 generates sounds by converting the instruction data of the breath action output from the voice instruction data output unit 24 into mechanical vibrations of a membrane. The speaker 25 instructs the breath action by voice guidance according to the instruction data. For example, when instructing an exhalation action, the speaker 25 outputs a voice sound of “Breathe out”. When instructing an inhalation action, the speaker 25 outputs a voice sound of “Breathe in”. Further, when instructing an action of a breath held condition (state maintenance action), the speaker 25 outputs the voice sound of “Hold a breath”.

As shown in FIG. 3A, the speaker 25 outputs voice sound in the order of “Breathe in” and “Hold a breath” in the case of the instruction data “a” corresponding to “Chest and Examination A”, for example. The speaker 25 outputs voice sound in the order of “Breathe in” and “Hold a breath” in the case of the instruction data “c” corresponding to “Abdomen and Examination C”, for example. The speaker 25 outputs voice sound of “Hold a breath” in the case of the instruction data “e” corresponding to “Chest and Examination E”, for example. In addition, the speaker 25 outputs voice sound in the order of “Breathe out” and “Hold a breath” in the case of the instruction data “f” corresponding to “Lung and Examination F”, for example.

The display 26 instructs a breath action by a visual display in accordance with instruction data. That is, the display 26 displays the instruction data of the breath action output from the control unit 21 in the form of, for example, characters, symbols, numbers, figures, or the combination thereof. The display 26 is composed of, for example, a liquid crystal display, an organic EL display, an LED (light emitting diode) display, or a VF (vacuum fluorescent) display.

For example, when instructing an exhalation action, the display 26 displays “Breathe out”. Further, when instructing an inhalation action, the display 26 displays “Breathe in”. When instructing an action of a breath held state (state maintenance action), the display 26 displays “Hold a breath”.

As shown in FIG. 3A, the display 26 displays breath actions in the order of “Breathe in” and “Hold a breath” in the case of the instruction data “a” corresponding to “Chest and Examination A”, for example. The display 26 displays breath actions in the order of “Breathe in” and “Hold a breath” in the case of the instruction data “c” corresponding to “Abdomen and Examination C”, for example. The display 26 displays a breath action of “Hold a breath” in the case of the instruction data “e” corresponding to “Chest and Examination E”, for example. The display 26 displays breath actions in the order of “Breathe out” and “Hold a breath” in the case of the instruction data “f” corresponding to “Lung and Examination F”, for example. The display of breath actions by the display 26 is performed at the same time as the voice sound output by the speaker 25.

The voice generation device 20 corresponds to the action instruction device of the present invention. The table 22 corresponds to the instruction data storage unit of the present invention. At least one of the speaker 25 and the display 26 corresponds to the instruction execution unit of the present invention. The operation unit 23 corresponds to the action instruction operation unit of the present invention. The operation display 23A corresponds to the display unit of the present invention. The RIS corresponds to the external management system of the present invention.

As shown in FIG. 2, the input port 10, the image processing unit 11, the console 12, the exposure switch 14, the storage unit 15, the control unit 21 of the voice generation device 20, the table 22, the operation unit 23, and the instruction data output unit 24 of the X-ray imaging apparatus 1 are provided in an operation room. The X-ray tube 2, the FPD 3, the column 4 (see FIG. 1), the upright stand 5 (see FIG. 1), the imaging control unit 13, the speaker 25 of the voice generation device 20, and the display 26 are provided in the X-ray room. A technician in the operation room performs imaging by remotely operating the X-ray tube 2, the FPD 3, the column 4, the upright stand 5, etc., provided in the X-ray room.

### <Operation of X-ray Imaging Apparatus 1>

Next, the operation of the X-ray imaging apparatus 1 will be described with reference to FIG. 1 to FIG. 4.

When the input port 10 receives an examination order from the RIS, the examination order is stored in the storage unit 15. The examination order includes, for example, the information about the subject M1 (name, sex, age, etc.) and two X-ray imaging menus (see FIG. 3B). The first X-ray imaging menu includes information about “Chest and Examination A”. The second X-ray imaging menu includes information about “Chest and Examination B”. The storage unit 15 is storing a plurality of examination orders including the received examination order.

The technician selects any examination order from the plurality of examination orders by using the operation display 12A and the operation input unit 12B of the console 12. For example, when the examination order relating to the subject M1 is selected, the imaging condition corresponding to each of the two X-ray imaging menus of the selected examination order is read out from the storage unit 15. The examination order on the subject M1 and the imaging condition corresponding to each X-ray imaging menu are hierarchically displayed on the operation display 12A in a tree structure. The technician makes the required changes to the imaging condition by using the operation display 12A and the operation input unit 12B. The examination order on the subject M1 may be newly generated by using the operation display 12A and the operation input unit 12B.

The selected examination order is sent to the control unit 21 of the voice generation device 20. As described above, the selected examination order relates to, for example, the subject M1. The examination order for the subject M1 includes two X-ray imaging menus as shown in FIG. 3B. The first X-ray imaging menu includes information about “Chest and Examination A”. The second X-ray imaging menu includes information about “Chest and Examination B”.

Upon receipt of the combination of “Chest and Examination A”, the control unit 21 acquires the instruction data “a” corresponding to the received combination of “Chest and Examination A” from the table 22. Similarly, when the control unit 21 receives the combination of “Chest and Examination B”, it acquires the instruction data “b” corresponding to the received combination of “Chest and Examination B” from the table 22. That is, the control unit 21 acquires from the table 22 two instruction data “a”, “b” corresponding to the combination of the imaging region (e.g., chest) and the examination type (e.g., Examination A or Examination B) of the two X-ray imaging menus in order to perform X-ray imaging based on the two X-ray imaging menus in order.

In the voice generation device 20, the operation display 23A of the operation unit 23 displays a part or all of the contents of the X-ray imaging menu (the examination type and the imaging region as shown in FIG. 4). The operation display 23A also displays the instruction data. In FIG. 4, for example, the name of the subject M1, the chest (imaging region), and the name of Examination A (examination type) are displayed on the operation display 23A. Note that the operation display 23A displays the inhalation action and the state maintenance action in this order as the instruction data “a” of breath actions. The second X-ray imaging menu is displayed by switching to another window.

The technician can change the instruction data “a” (breath actions) displayed on the operation display 23A of the operation unit 23 using the operation input unit 23B. In operating the voice generation device 20, the technician can confirm the imaging region and the examination type, as well as the breath action instruction data. Therefore, manual change of the breath action instruction data by the technician can be easily performed. The breath action instruction data is acquired by the control unit 21 according to the combination of the imaging region and the examination type. The technician is not required to newly generate breath action instruction data, and even in cases where necessary changes of the breath action instruction data have been made, the changes can be relatively minimized. Therefore, it is easy to set breath action instruction data while preventing the occurrence of setting errors.

Positioning is performed by moving the FPD 3 (see FIG. 1 and FIG. 2) vertically so as to be positioned in the imaging region of the set imaging condition. The direction of the X-ray tube 2 is adjusted so that the X-rays from the X-ray tube 2 (see FIG. 1 and FIG. 2) are emitted to the FPD 3 positioned in the imaging region and the column 4 (see FIG. 1) is expanded and contracted so that the X-ray tube 2 is positioned at a position opposite to the imaging region. The subject M is supported in a standing posture with the subject M of the standing posture (see FIG. 1) facing to the upright stand 5 (see FIG. 1). Note that if the positioning of the X-ray tube 2 and the FPD 3 do not require much time, the positioning of the X-ray tube 2 and the FPD 3 may be performed after the subject M is supported in the standing posture.

The technician presses the exposure switch 14 (see FIG. 2) halfway to apply a tube voltage to the X-ray tube 2 to set the irradiation preparation. Then, the technician fully presses the exposure switch 14 to initiate the X-ray irradiation from the X-ray tube 2. Note that if it takes a long time to prepare for the irradiation, the exposure switch 14 may be pressed halfway in advance before the input operation to the console 12 or the positioning.

### [X-Ray Imaging By First X-Ray Imaging Menu]

X-ray imaging is performed on the subject M1 based on the first X-ray imaging menu. When the exposure switch 14 is fully pressed, the control unit 21 of the voice generation device 20 transmits instruction data “a” to the speaker 25 and the display 26. Specifically, the control unit 21 transmits the instruction data “a” for voice sound to the speaker 25 via the instruction data output unit 24 to perform voice guidance. At the same time, the control unit 21 transmits instruction data “a” for displaying to the display 26 to perform visual display.

The speaker 25 outputs voice sound in the order of “Breathe in” and “Hold a breath” according to the instruction data “a”. In accordance with the voice guidance by the speaker 25, the display 26 displays breath actions in the order of “Breathe in” and “Hold a breath” according to the instruction data “a”. Thereafter, the X-ray tube 2 emits X-rays. The FPD 3 detects X-rays irradiated from the X-ray tube 2 and transmitted through the subject M1. The image processing unit 11 processes the image of pixel values of X-rays detected by the FPD 3 to generate an X-ray image.

### [X-Ray Imaging By Second X-Ray Imaging Menu]

After performing X-ray imaging by the first X-ray imaging menu, X-ray imaging is performed by the second X-ray imaging menu. At the timing when the exposure switch 14 is fully pressed, the control unit 21 of the voice generation device 20 transmits the instruction data “b” to the speaker 25 and the display 26.

The speaker 25 outputs voice sound in the order of “Breathe out” and “Hold a breath” according to the instruction data “b”. In accordance with the voice guidance of the speaker 25, the display 26 displays breath actions in the order of “Breathe out” and “Hold a breath” according to the instruction data “a”. Thereafter, the X-ray tube 2 emits X-rays to produce an X-ray image. In this manner, X-ray imaging by the two X-ray imaging menus is performed on the subject M1.

The display of breath actions by the display 26 is performed at the same time as the output of the voice sound of the breath actions by the speaker 25. Thereby, the subject M1 can accurately grasp the audio instruction by the speaker 25, and can accurately grasp the instruction by the display 26.

According to this embodiment, in the voice generation device 20 and the X-ray imaging apparatus 1, the table 22 stores a plurality of pieces of instruction data corresponding to a plurality of combinations of an imaging region and an examination type, respectively. The speaker 25 and the display 26 instruct breath actions according to instruction data. The instruction data corresponding to a combination of an imaging region and an examination type is acquired by the control unit 21 from the table 22. Thus, it is possible to prevent a setting error of breath actions corresponding to a predetermined combination of an imaging region and an examination type. Therefore, it is possible to prevent X-ray imaging from being performed again due to an incorrect setting, which in tun can prevent the burden on the subject. Further, it is possible to prevent the examination time from being extended by repeating X-ray imaging, which in turn can prevent the throughput from being lowered.

The speaker 25 instructs breath actions by voice guidance in accordance with the instruction data acquired by the control unit 21. According to the voice guidance by the speaker 25, the subject M can perform breath actions. The display 26 instructs breath actions by visual display in accordance with the instruction data acquired by the control unit 21. According to the visual display of the display 26, the subject M can perform breath actions.

Further, the voice generation device 20 and the X-ray imaging apparatus 1 is provided with the operation display 23A for displaying a part or all of the imaging region and the examination type, as well as instruction data, and the operation unit 23 having an operation input unit 23B capable of changing the instruction data displayed on the operation display 23A.

In operating the voice generation device 20, the technician can confirm the imaging region and the examination type as well as instruction data. Therefore, the manual change of the instruction data can be easily performed by the technician. The instruction data is acquired by the control unit 21 according to a combination of an imaging region and an examination type. The technician is not required to newly generate instruction data, and even if changes required for breath action instruction data are performed, the change can be relatively minimized. Therefore, it is possible to easily set breath action instruction data while preventing the occurrence of setting errors.

Although the imaging region display (monitor) is provided on the side of the X-ray imaging apparatus side, the commands (states of an exhalation, an inhalation, a breath holding) for instructing an exhalation, an inhalation, and a breath holding is displayed on the side of the operation panel of the medical-use voice generation device 20. Therefore, there is a problem that the setting is likely to be erroneously set at the time of the operation by the technician. In this respect, since the technician can confirm the breath action instruction data by the operation display 23A of the operation unit 23, it is possible to prevent setting errors from being occurred.

The input port 10 receives a plurality of X-ray imaging menus sent from the RIS. The plurality of X-ray imaging menus each includes an imaging region and an examination type. The control unit 21 acquires instruction data corresponding to a combination of an imaging region and an examination type of the plurality of X-ray imaging menus from the table 22 in order to perform X-ray imaging in order based on the plurality of X-ray imaging menus. This makes it possible to acquire instruction data of the optimum breath action for each combination of an imaging region and an examination type. It is possible to prevent a setting error of breath actions corresponding to a plurality of combinations of imaging region and examination type from being occurred.

### Embodiment 2

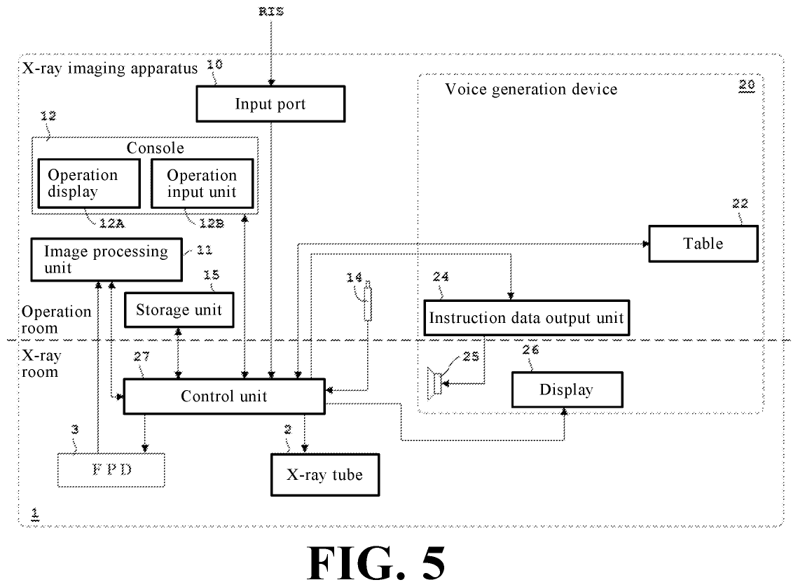

Next, with referring to the attached drawings, Embodiment 2 of the present invention will be described. FIG. 5 is a block diagram of an X-ray imaging apparatus and a voice generation device according to Embodiment 2.

In the above-described Embodiment 1, the X-ray imaging apparatus 1 is provided with the voice generation device 20 as an external device. In contrast, in Embodiment 2, the X-ray imaging apparatus 1 is provided with a voice generation device 20 inside thereof. That is, the X-ray imaging apparatus 1 is provided with a built-in voice generation device 20. Therefore, in Embodiment 1, as shown in FIG. 2, the imaging control unit 13 and the control unit 21 are individually provided. In this respect, in Embodiment 2, as shown in FIG. 5, the imaging control unit 13 (see FIG. 2) and the control unit 21 (see FIG. 2) are composed of one control unit 27. Therefore, upon receipt of a combination of an imaging region and an examination type from the input port 10 or the operation input unit 12B of the console 12, the control unit 27 acquires instruction data corresponding to the received combination from the table 22.

Further, in Embodiment 1, as shown in FIG. 2, the operation unit 23 is provided separately from the console 12. In this regard, in Embodiment 2, the console 12 may be configured to have the function of the operation unit 23. In this instance, the operation display 12A of the console 12 displays a part or all of the contents of X-ray imaging menus (examination type, imaging region, etc.), as shown in FIG. 4. The operation display 12A also displays instruction data. The screen shown in FIG. 4 and the screen for setting imaging conditions, etc. can be arbitrarily switched. In FIG. 5, the table 22 is provided separately from the storage unit 15. In this regard, the storage unit 15 may have the table 22.

Since the operation of the X-ray imaging apparatus 1 of Embodiment 2 is the same as that of Embodiment 1 described above, the description will be omitted. Note that the console 12 corresponds to the action instruction operation unit of the present invention.

According to this embodiment, the table 22 stores a plurality of pieces of breath action instruction data corresponding to a plurality of combinations of an imaging region and an examination type. The speaker 25 and the display 26 instruct breath actions according to instruction data. The instruction data corresponding to the combination of imaging region and examination type is acquired by the control unit 27 from the table 22. Thus, it is possible to prevent a setting error of breath actions corresponding to a predetermined combination of an imaging region and an examination type from being occurred. Therefore, it is possible to prevent X-ray imaging from being performed again due to an incorrect setting, which in turn can prevent the burden on the subject. Further, it is possible to prevent the examination time from being extended by repeating X-ray imaging, which in turn can prevent the throughput from being lowered.

The present invention is not limited to the above embodiments, and can be modified as described below.

(1) In the above-described embodiments, a digital X-ray detector, such as a flat-panel X-ray detector (FPD), is used as an X-ray detector, but an analog X-ray detector, such as an image intensifier (I.I.) and an X-ray film, may be used.

(2) In each of embodiments and the modification (1) described above, the medical voice generation device is provided with a display 26 for instructing breath actions by relatively displaying, but it is not always necessary to provide a display 26. A display is optional (addition other than a standard specification).

(3) In each embodiment and the modification (1) described above, the medical voice generation device is provided with the speaker 25 for instructing breath actions by voice guidance, but it is not always necessary to provide the speaker 25.

(4) In cases where the X-ray imaging apparatus is provided with a medical-use voice generation device as in Embodiment 2 described above, the imaging control unit 13 (see FIG. 2) for performing overall control for imaging and the auto voice control unit 21 (see FIG. 2) for performing control for outputting preset signals corresponding to an imaging region are composed of one control unit 27 (see FIG. 5). However, the former imaging control unit 13 and the latter control unit 21 need not necessarily be one. Even in cases where the X-ray imaging apparatus is provided with the medical-use voice generation device as in Embodiment 2 described above, the former imaging control unit 13 and the latter control unit 21 may be separately provided as in Embodiment 1 described above.

(5) In each embodiment and modification described above, the input port 10 is composed of, for example, a terminal and an electric circuit, etc. In this regard, the input port 10 may be composed of a DR system (image processing unit). The DR system may be composed of a computer, such as, e.g., a personal computer and a workstation. When the DR system receives an imaging order from the RIS, the imaging order is stored in a storage unit of the DR system. When an arbitrary imaging order is selected from a plurality of imaging orders stored in the storage unit, the imaging condition corresponding to the subject and the X-ray imaging menu is notified of the imaging control unit 13.

(6) In each embodiment and modified embodiment described above, the control unit 21 acquires instruction data corresponding to an imaging region and an examination type from table 22. The voice generation device 20 may be configured to invalidate the instruction data acquired by the control unit 21 and instruction data may be manually sent to the speaker 25 and the display 26.

Specifically, the voice generation device 20 is provided with at least three switches and a manual selector switch. The three switches are an exhalation action instruction switch, an inhalation action indication switch, and a state maintenance action instruction switch. The exhalation action instruction switch outputs instruction data for performing an exhalation action. The inhalation action instruction switch outputs instruction data for performing an inhalation action. The state maintenance action instruction switch outputs instruction data for performing a state maintenance action. The manual selector switch disables instruction data acquired by the control unit 21 or enables instruction data acquired by the control unit 21. When the instruction data is disabled, the instruction data from the three switches is enabled.

The technician selects one of the three switches. For example, the inhalation action instruction switch and the state maintenance action instruction switch are selected in this order. Thus, for example, the speaker 25 outputs voice sound in the order of “Breathe in” and “Hold a breath”.

The manual selector switch may be configured to not acquire instruction data by referring to the table 22, or may be configured to acquire instruction data by referring to the table 22. When the instruction data is not acquired by referring to the table the 22, it may be configured to enable the instruction data from the three switches.

(7) In each embodiment and modified embodiment described above, the voice sound has three patterns of “Breathe in”, “Breathe out”, and “Hold a breath as it is”. In this regard, other voice sounds, such as “Deeply breathe in”, may be added thereto.

1. An action instruction device comprising:
an instruction data storage unit configured to store a plurality of pieces of breath action instruction data corresponding to a plurality of combinations of an imaging region and an examination type, respectively, the plurality of pieces of breath action instruction data each including at least one of an exhalation action, an inhalation action, and a state maintenance action;
a control unit configured to acquire the breath action instruction data corresponding to a received combination of the imaging region and the examination type upon receipt of the combination of the imaging region and the examination type from outside; and
an instruction execution unit configured to instruct a breath action in accordance with the breath action instruction data acquired by the control unit.

2. The action instruction device as claimed in claim 1,
wherein the instruction execution unit is a speaker configured to instruct the breath action by voice guidance in accordance with the breath action instruction data.

3. The action instruction device as claimed in claim 1,
wherein the instruction execution unit is a display configured to instruct the breath action by a visual display in accordance with the breath action instruction data.

4. The action instruction device as claimed in claim 1, further comprising:
an action instruction operation unit including a display unit configured to display a part or all of the imaging region and the examination type and also display the breath action instruction data and an operation input unit capable of changing the action instruction data displayed on the display unit.

5. An X-ray imaging apparatus for performing X-ray imaging, comprising:
an input unit configured to input a combination of an imaging region and an examination type;
an X-ray irradiation unit configured to emit X-rays;
an X-ray detector configured to detect X-rays;
an image processing unit configured to process an image composed of a pixel value of X-rays detected by the X-ray detector to generate an X-ray image; and
an action instruction device configured to instruct a breath action to a subject,
wherein the action instruction device includes:
an instruction data storage unit configured to store a plurality of pieces of breath action instruction data corresponding to a plurality combinations of an imaging region and an examination type, respectively, the plurality of pieces of breath action instruction data each including at least one of an exhalation action, an inhalation action, and a state maintenance action;
a control unit configured to acquire the breath action instruction data corresponding to a received combination upon receipt of the combination of the imaging region and the examination type from the input unit; and
an instruction execution unit configured to instruct a breath action according to the breath action instruction data acquired by the control unit.

6. The X-ray imaging apparatus as claimed in claim 5,
wherein the input unit receives a plurality of X-ray imaging menus sent from an external management system, the plurality of X-ray imaging menus each including the imaging region and the examination type, and
wherein the control unit acquires the breath action instruction data corresponding to the combination of the imaging region and the examination type of the plurality of X-ray imaging menus from the breath action storage unit in order to sequentially perform X-ray imaging based on the plurality of X-ray imaging menus.
